(19) United States
(12) Patent Application Publication
(10) Pub. No.: US 2007/0083054 A1
(43) Pub. Date: Apr. 12, 2007

(54) Novel amides of lipoic acid

(76) Inventors: Lowell J. Lawrence, Lexington, KY (US); Rodney L. Eisenberg, Richmond, KY (US)

Correspondence Address: KING & SCHICKLI, PLLC, 247 NORTH BROADWAY, LEXINGTON, KY, 40507, US

(21) Appl. No.: 11/248,358

(22) Filed: Oct. 12, 2005

(51) Int. Cl. C07D 339/04

(52) U.S. Cl. 549/35

(57) ABSTRACT

A process is provided for producing lipoate amides from α-lipoic acid. The process comprises reacting α-lipoic acid with a primary or secondary amine in the presence of dicyclohexylcarbodiimide and N-hydroxysuccinimide.

### TECHNICAL FIELD

This invention generally relates to processes for the production of amides of thioctic acid/lipoic acid.

### BACKGROUND OF THE INVENTION

Thioctic acid, also known as α-lipoic acid, is well known in the art. Lipoic acid and its derivatives have a number of uses including but not limited to the treatment of liver disease, as an antidote to poisonous mushrooms, the treatment of diabetes, the treatment of asthma and as an antioxidant.

The preparation of lipoic acid amides is disclosed in, for example, U.S. Pat. No. 3,223,712. In one possible amidation reaction of lipoic acid, lipoic acid is dissolved in a suitable organic solvent and the resulting solution is reacted with alkyl chlorocarbonate in the presence of a tertiary lower alkyl amine at a temperature of less than 0° C. to form the corresponding mixed carbonic-carboxylic acid anhydride. The resulting anhydride is then reacted with ammonia or the aqueous organic solvent solution thereof at a temperature below room temperature, preferably below 0° C. to obtain the desired lipoic acid amide. In another approach lipoic acid is reacted directly with urea to obtain the lipoic acid amide in one step. In still another approach lipoic acid is amidated with a combination of aqueous or alcoholic ammonia with a known acetylating agent such as acetic anhydride or acetyl chloride.

The present invention relates to a new method for preparing lipoic acid amides and to novel chemical compounds prepared by that method.

### SUMMARY OF THE INVENTION

In accordance with the purposes of the present invention as described herein, a process is provided for producing lipoate amides from α-lipoic acid. This is applicable not only to racemic but also to both isolated S-isomer and R-isomer. The process comprises reacting α-lipoic acid with a primary or secondary amine in the presence of dicyclohexylcarbodiimide and N-hydroxysuccinimide to produce a lipoate amide. In one possible embodiment of the invention the primary amine is N,N-dimethylethylene diamine.

In addition the method includes reacting the resulting lipoate amide with 4,7,10-trioxa-1,13-tridecandiamine to produce a polyethylene glycol-derived amino acid such as N-(3-(2-(2-(3-aminopropoxy)ethoxy)ethoxy)propyl)-5-(1,2-dithiolan-3-yl)pentanamide.

In accordance with yet another aspect of the present invention a compound is provided comprising 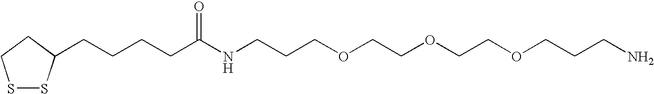
N-(3-(2-(2-(3-aminopropoxy)ethoxy)ethoxy)propyl)-5-(1,2-dithiolan-3-yl)pentanamide.

### DETAILED DESCRIPTION OF THE INVENTION

The present invention relates to a process for producing lipoate amides from α-lipoic acid. The process may be broadly described as comprising the step of reacting α-lipoic acid with dicyclohexylcarbodiimide, N-hydroxysuccinimide and a primary or secondary amine to produce a lipoate amide. The lipoic acid is first dissolved in an appropriate solvent such as anhydrous dichloromethane and then stirred at a room temperature under an inert atmosphere. The dicyclohexylcarbodiimide is similarly dissolved in an appropriate solvent such as dichloromethane and added to the lipoic acid solution. The resulting mixture is stirred and a solution of N-hydroxysuccinimide dissolved in acetonitrile is added. After stirring the primary or secondary amine is added neat to the solution and the reaction is stirred overnight at room temperature. This reaction is illustrated by the following equation where the primary amine is N,N-dimethylethylene diamine.

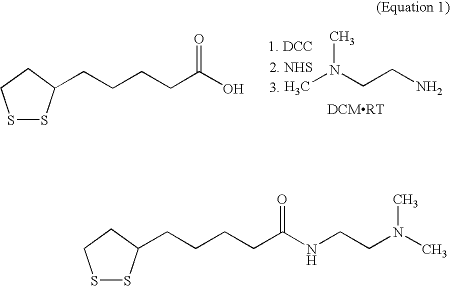

In the reaction mixture the dicyclohexylcarbodiimide or other carbodiimide functions as an activator of the carboxylic acid functional group. In order to be effective, it should be present in an amount of between about 1.0 and about 1.5 molar equivalents. Further the N-hydroxysuccinimide functions as an intermediate carboxylic acid activator. In order to be effective, it should be present in an amount of between about 1.0 and about 1.5 molar equivalents.

When the primary amine used is 4,7,10-trioxa-1,13-tridecandiamine, it is possible to produce the compound N-(3-(2-(2-(3-aminopropoxy)ethoxy) ethoxy)propyl)-5-(1,2-dithiolan-3-yl)pentanamide having the structural formula:

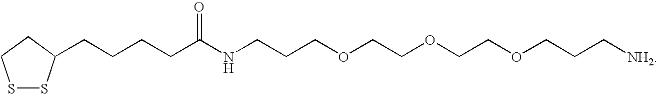

This is done by reacting the product of Equation 1 with 4,7,10-trioxa-1,13-tridecandiamine. The reaction is illustrated in the following equation:

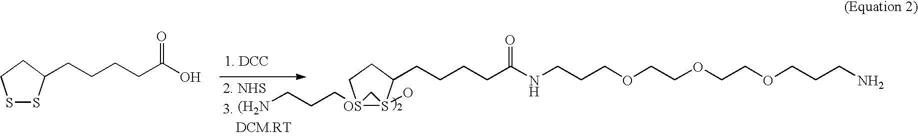

Cysteine may be added after the reaction is complete in order to stabilize the resulting lipoic acid amide and prevent polymerization. The cysteine may be added in an amount of between about 0.5 and 5%.

The following synthesis and examples are presented to further illustrate the invention, but it is not to be considered as limited thereto.

### EXAMPLE 1

Lipoic acid (5 g) was dissolved in anhydrous dichloromethane (DCM, 200 mL) and stirred at room temperature under an inert atmosphere.

Dicyclohexylcarbodiimide (DCC, 6.0 g) was dissolved in DCM (10 mL) and added to the lipoic acid solution and the resulting mixture stirred for 30 min. A solution of N-hydroxysuccinimide (NHS, 3.35 g) was dissolved in acetonitrile (5 mL) and added to the solution. After stirring for 30 minutes, N,N-dimethylethylene diamine (6.5 g) was added neat and the reaction stirred overnight at room temperature.

The reaction was filtered and washed three times with an aqueous solution consisting of 1M NaOH and 1 M NaCl, dried over MgSO₄ and evaporated under reduced pressure. The resulting solid was dissolved in 1M HCl (200 mL), filtered and the aqueous solution added to chloroform (200 mL). The pH was adjusted to 12-14 with 50% NaOH and the two phase system mixed thoroughly. The phases were separated, and the organic layer dried with MgSO₄, filtered and the volatiles removed under reduced pressure. The resulting N-(2-(dimethylamino)ethyl)-5-(1,2-dithiolan-3-yl)pentanamide, an oil, was stored at less than −5° C. Cysteine could be added after the filtering step to stabilize the reaction product.

### EXAMPLE 2

Lipoic acid (5 g) was dissolved in anhydrous dichloromethane (DCM, 200 mL) and stirred at room temperature under an inert atmosphere.

Dicyclohexylcarbodiimide (DCC, 6.0 g) was dissolved in DCM (10 mL) and added to the lipoic acid solution and the resulting mixture stirred for 30 min. A solution of N-hydroxysuccinimide (NHS, 3.35 g) was dissolved in acetonitrile (5 mL) and added to the solution. After stirring for 30 minutes, 4,7,10-trioxa-1,13-tridecandiamine (16.0 g) was added neat and the reaction stirred overnight at room temperature.

The reaction was filtered, and washed three times with an aqueous solution consisting of 1M NaOH and 1 M NaCl, dried over MgSO₄ and evaporated under reduced pressure. The solid was dissolved in 1M HCl (200 mL), filtered and the aqueous solution added to chloroform (200 mL). The pH was adjusted to 12-14 with 50% NaOH and the two phase system mixed thoroughly. The phases were separated, and the organic layer dried with MgSO₄, filtered and the volatiles removed under reduced pressure. The resulting N-(3-(2-(2-(3-aminopropoxy)ethoxy)ethoxy)propyl)-5-(1,2-dithiolan-3-yl)pentanamide, an oil, was stored at less than −5° C. Cysteine could be added after the filtering step to stabilize the reaction product.

In summary, numerous benefits have been described which result from employing the concepts of the present invention. Advantageously, the present process allows one to more efficiently prepare lipoate amides from α-lipoic acid quickly and in high yields. The invention has been described herein with reference to certain preferred embodiments. Obvious variations and modifications thereof will become apparent to those skilled in the art and, accordingly, the invention is not to be considered as being limited thereto.

What is claimed:

1. A process for producing lipoate amides from α-lipoic acid, comprising:
reacting α-lipoic acid with a primary or secondary amine in the presence of dicyclohexylcarbodiimide and N-hydroxysuccinimide to produce a lipoate amide.

2. The process of claim 1 wherein said primary or secondary amine is dimethylethylene diamine.

3. The process of claim 2, further including reacting said lipoate amide with 4,7,10-trioxa-1,13-tridecandiamine to produce a polyethylene glycol-derived amino amide.

4. The process of claim 1, further including reacting said lipoate amide with 4,7,10-trioxa-1,13-tridecandiamine to produce a polyethylene glycol-derived amino amide.

5. The process of claim 3 including adding cysteine to the lipoate amide.

6. The process of claim 4 including adding cysteine to the lipoate amide.

7. The process of claim 1 including adding cysteine to the lipoate amide.

8. A compound, comprising: [chemical structure]
